(19) United States
(12) Patent Application Publication
(10) Pub. No.: US 2025/0366721 A1
(43) Pub. Date: Dec. 4, 2025

(54) WEARABLE DEVICE

(71) Applicant: LG ELECTRONICS INC., Seoul (KR)

(72) Inventor: Byungjoon RHEE, Seoul (KR)

(73) Assignee: LG ELECTRONICS INC., Seoul (KR)

(21) Appl. No.: 18/874,955

(22) PCT Filed: Jun. 14, 2022
(86) PCT No.: PCT/KR2022/008369

§ 371 Date: Dec. 13, 2024

(51) Int. Cl. A61B 5/0205; A61B 5/00; A61B 5/0225; A61B 5/145; A61B 5/1455

(52) U.S. Cl. CPC A61B 5/0205; A61B 5/0004; A61B 5/14532; A61B 5/1455; A61B 5/681; A61B 5/742; A61B 5/0225; A61B 2562/0204

(57) ABSTRACT

A wearable device may include a plurality of blocks on a first surface of a first substrate, and a plurality of connecting wires configured to connect the plurality of blocks. The plurality of blocks may each include a second substrate on the first substrate, a light detector on the second substrate, and a plurality of pixels around the light detector on the second substrate. The plurality of pixels may each include at least one or more first semiconductor light-emitting element configured to emit first light, at least one or more second semiconductor light-emitting element configured to emit second light, and at least one or more third semiconductor light-emitting element configured to emit third light. The second semiconductor light-emitting element may emit a portion of the second light forward and another portion of the second light backward to be transmitted to the light detector.

### TECHNICAL FIELD

The embodiment relates to a wearable device.

### BACKGROUND ART

Healthcare technology is receiving much attention due to social issues such as rapid entry into an aging society and the resulting increase in medical expenses. Accordingly, a wearable device, which is small medical devices that can be carried by individuals as well as medical devices that can be used in hospitals or testing institutions, is being developed. A wearable devices has the advantage of being able to measure biological signal from users regardless of location and time.

### DISCLOSURE

### Technical Problem

The embodiment provides a wearable device that is portable and can measure biological signal.

The embodiment provides a wearable device that can increase the accuracy of biological signal measurement and improve reliability.

The embodiment provides a wearable device that comprises a display and a light source that can measure at the same time.

The embodiment provides a wearable device that can be miniaturized.

The embodiment provides a wearable device capable of measuring various biological signals.

The embodiment provides a wearable device having a stretchable property.

The embodiment provides a wearable device capable of communicating with an external device.

### Technical Solution

According to one aspect of the embodiment, a wearable device, comprising: a first substrate; a plurality of blocks on a first surface of the first substrate; and a plurality of connection wirings configured to connect between the plurality of blocks, wherein the plurality of blocks each comprises: a second substrate on the first substrate; a light detector on the second substrate; and a plurality of pixels around the light detector on the second substrate, wherein the plurality of pixels each comprises: at least one or more first semiconductor light-emitting element configured to emit a first light; at least one or more second semiconductor light-emitting element configured to emit a second light; and at least one or more third semiconductor light-emitting element configured to emit a third light, and wherein the second semiconductor light-emitting element is configured to emit a portion of the second light forward and another portion of the second light backward to be transmitted to the light detector.

The first semiconductor light-emitting element may emit a portion of the first light forward and another portion of the first light backward to be transmitted to the light detector.

The third semiconductor element may emit a portion of the third light forward, and another portion of the third light backward to be transmitted to the light detector.

An image may be displayed by the first light, the second light, and the third light emitted forward, and at least one or more of the first light, the second light, or the third light emitted backward may be received as a first blood pressure signal by the light detector through a subject.

The second substrate and the light detector may each have a square shape, and the size of the light detector may be smaller than the size of the second substrate. The plurality of pixels may be positioned around the corners of the light detector.

The plurality of blocks may each comprise a plurality of fourth semiconductor light-emitting elements configured to emit fourth light around the light detector on the second substrate. The fourth light may be received as a glucose signal by the light detector through a subject. The plurality of fourth semiconductor light-emitting elements may be positioned between the plurality of pixels.

The first to fourth semiconductor light-emitting elements may each have a size of micrometer or less.

The wearable device may comprise a control module on one side of the first substrate.

The wearable device may comprise: an expansion/contraction member on a second side of the first substrate opposite the first side; a pressure adjustment part configured to adjust a pressure of the expansion/contraction member so that pressure is applied to a subject; and an acoustic sensor configured to detect a second blood pressure signal generated from the subject.

Blood pressure information obtained based on the first blood pressure signal and the second blood pressure signal may be displayed on the plurality of pixels.

The wearable device may comprise: a communication unit configured to transmit the second blood pressure signal to an external device.

The first substrate comprises a stretchable substrate.

The wearable device may comprise a patch type or a cuff type.

According to another aspect of the embodiment, a wearable device, comprises: a first substrate; a display unit comprising a plurality of blocks on the first substrate; a plurality of connection wirings configured to connect between the plurality of blocks; and a biological signal measuring unit on one side of the display unit on the first substrate, wherein the plurality of blocks each comprises: a second substrate on the first substrate; and a pixel on the second substrate, wherein the pixel comprises: at least one first or more semiconductor light-emitting element configured to emit a first light; at least one or more second semiconductor light-emitting element configured to emit a second light; and at least one or more third semiconductor light-emitting element configured to emit a third light, wherein the biological signal measuring unit comprises: a second substrate on the first substrate; a light detector on the second substrate; and a plurality of fourth semiconductor light-emitting elements configured to emit a fourth light around the light detector on the second substrate, and wherein the fourth light is identical to light emitted from one of the first to third semiconductor light-emitting elements.

The fourth light may be received as a blood pressure signal by the light detector through a subject.

The biological signal measuring unit may comprise a plurality of fifth semiconductor light-emitting elements configured to emit fifth light around the light detector on the second substrate.

The fifth light may be received as a glucose signal by the light detector through a subject.

The first to fifth semiconductor light-emitting elements may each have a size of micrometer or less.

### Advantageous Effects

According to an embodiment, as illustrated in FIGS. 2 to 5, a plurality of blocks 120 are included, each of the plurality of blocks 120 comprises a light detector 128 and a plurality of pixels PX1 to PX4, each of the plurality of pixels PX1 to PX4 comprises a plurality of semiconductor light-emitting elements, and at least one or more semiconductor light-emitting element among the plurality of semiconductor light-emitting elements forms a biological signal measuring unit together with the light detector 128, thereby enabling image display and biological signal measurement. That is, since the display and the sensor are integrated, there is no need to provide a separate sensor, so that compact and lightweight design is possible.

According to an embodiment, as illustrated in FIG. 1, a wearable device 100 is configured in a patch type, so that it is portable and can be attached to the subject when needed to measure biological signal.

According to an embodiment, a plurality of blocks 120 capable of biological signal measurement may be disposed over a wide area, so that a plurality of biological signals can be measured from a wide surface of a subject, and based on these plurality of biological signals, biological signals can be measured more accurately, thereby improving reliability.

According to an embodiment, a semiconductor light-emitting element having a size of less than a micrometer may be used as a light source for biological signal measurement, thereby enabling miniaturization.

According to an embodiment, blood pressure signals or glucose signals can be measured by a plurality of semiconductor light-emitting elements and one light detector 128, so that more efficient healthcare can be implemented through measurement of various biological signals.

According to an embodiment, biological signals measured by a plurality of semiconductor light-emitting elements and one light detector 128 can be transmitted to an external device for display, thereby enabling efficient information exchange.

According to an embodiment, since it may be configured in a patch or cuff type and may be continuously fixed and in contact with the subject, the accuracy of biological signal measurement can be further improved.

Meanwhile, for example, as illustrated in FIGS. 2 to 5, a first blood pressure signal can be obtained by using at least one of the first to third lights 151 to 153 emitted from a plurality of first to third semiconductor light-emitting elements 121 to 123. In addition, as illustrated in FIG. 7, a second blood pressure signal comprising a Korotkoff sound signal can be obtained through an acoustic sensor 230. Therefore, by obtaining blood pressure information by considering not only the first blood pressure signal but also the second blood pressure signal, more accurate blood pressure information can be provided, thereby improving the reliability of information or products.

### DESCRIPTION OF DRAWINGS

FIG. 1 illustrates a wearable device according to the first embodiment worn on a wrist.

FIG. 2 is a perspective view illustrating a wearable device according to the first embodiment.

FIG. 3 is a plan view illustrating a wearable device according to the first embodiment.

FIG. 4 is a cross-sectional view illustrating a wearable device according to the first embodiment.

FIG. 5 illustrates a display using a semiconductor light-emitting element and a biological signal measurement in a wearable device according to the first embodiment.

FIG. 6 illustrates a wearable device according to the second embodiment being worn on the forearm.

FIG. 7 is a perspective view illustrating a wearable device according to the second embodiment.

FIG. 8 is a plan view illustrating a wearable device according to the third embodiment.

FIG. 9 is a cross-sectional view illustrating pixels in a wearable device according to the third embodiment.

FIG. 10 is a cross-sectional view illustrating a biological signal measurement unit in a wearable device according to the third embodiment.

FIG. 11 is a plan view illustrating a display and a biological signal measurement in a wearable device according to the third embodiment.

FIG. 12 is a cross-sectional view showing a display and a biological signal measurement in a wearable device according to the third embodiment.

### MODE FOR INVENTION

Hereinafter, the embodiment disclosed in this specification will be described in detail with reference to the accompanying drawings, but the same or similar elements are given the same reference numerals regardless of reference numerals, and redundant descriptions thereof will be omitted. The suffixes ‘module’ and ‘unit’ for the elements used in the following descriptions are given or used interchangeably in consideration of ease of writing the specification, and do not themselves have a meaning or role that is distinct from each other. In addition, the accompanying drawings are for easy understanding of the embodiment disclosed in this specification, and the technical idea disclosed in this specification is not limited by the accompanying drawings. Also, when an element such as a layer, region or substrate is referred to as being ‘on’ another element, this comprises that there can be directly on the other element or be other intermediate elements therebetween.

The embodiment may comprise a biological signal measuring device, which can be mounted on various devices.

The biological signal measuring device can be mounted on various types of wearable devices. The wearable device of the embodiment refers to a device that can be worn regardless of the position of the body and can realize various electronic functions based on IT technology.

For example, the wearable device may comprise, but is not limited thereto, a watch-type device, a band-type device, a ring-type device, a belt-type device, a necklace-type device, a hairband-type device, a headphone-type device, a glasses-type device, a patch-type device, a cuff-type device, etc. that are worn on the wrist.

In addition, the biological signal measuring device may be mounted on a smartphone, an AR device, a VR device, a tablet PC, a notebook PC, etc.

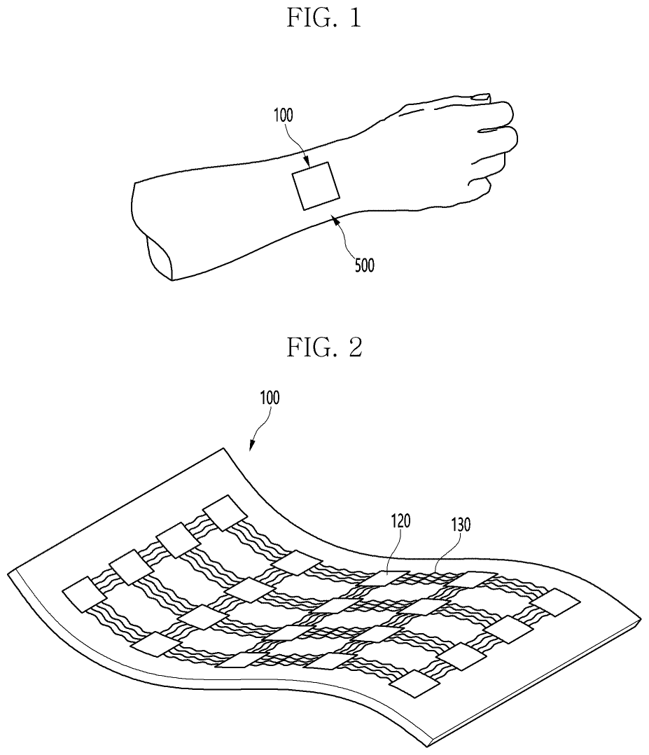

Hereinafter, various embodiments will be described with reference to FIGS. 1 to 12.

### FIRST EMBODIMENT

FIG. 1 illustrates a wearable device according to the first embodiment being worn on a wrist.

As illustrated in FIG. 1, the wearable device 100 according to the first embodiment may be worn on a wrist 500 and may measure various biological signal from the wrist 500. For example, the biological signal may comprise blood pressure, blood glucose, vascular age, arteriosclerosis, vascular elasticity, blood triglycerides, cardiac output, etc.

The wearable device 100 according to the first embodiment may be a patch-type device.

To this end, the patch-type device that comes into contact with the surface of the wrist 500 may have a layer or a surface-treatment surface comprising a material having an adhesive property. As described above, instead of the patch-type device, a watch-type device, a band-type device, a ring-type device, a belt-type device, etc. may be used.

Since the surface of the wrist 500 has a round surface or a curved surface, the wearable device 100 according to the first embodiment may have a layer comprising a material having a stretchable property, a flexible p property, a rollable property, etc. so as to be well attached to the wrist 500 having such a shape.

FIG. 2 is a perspective view illustrating a wearable device according to the first embodiment.

Referring to FIG. 2, the wearable device 100 according to the first embodiment may have a plurality of blocks 120 disposed within a substrate having a stretchable property. The plurality of blocks 120 may be connected to each other by a plurality of connection wirings 130.

For example, since the substrate has a material having a stretchable property, it can be stretched in a desired direction and can be freely bent. Accordingly, the entire surface of the wearable device 100 according to the first embodiment can easily come into surface contact with the surface of the wrist 500.

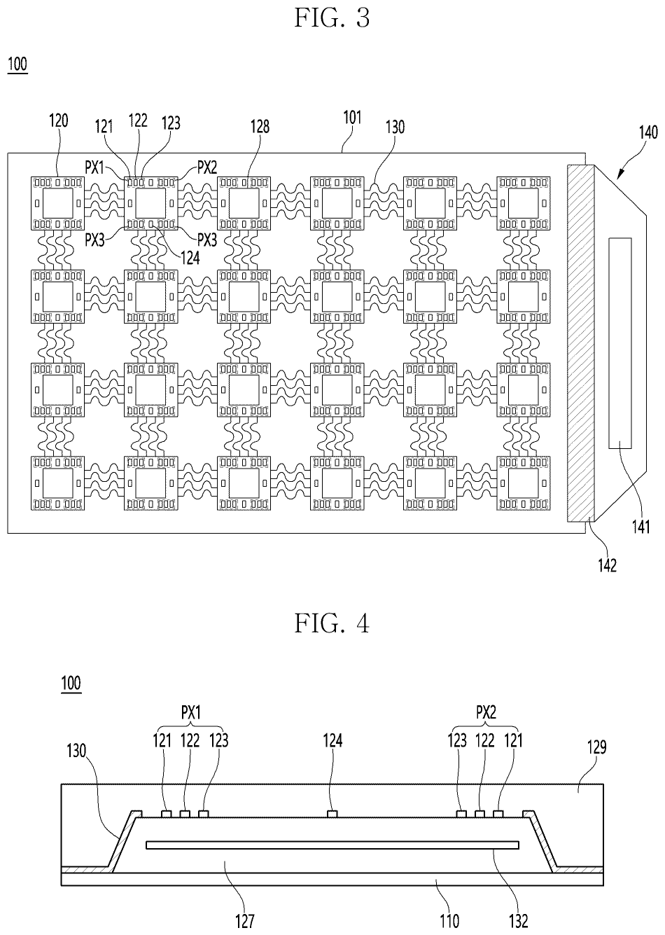

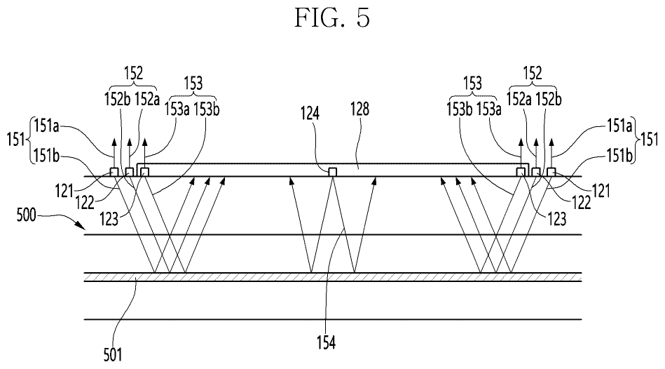

FIG. 3 is a plan view illustrating a wearable device according to the first embodiment. FIG. 4 is a cross-sectional view illustrating a wearable device according to the first embodiment.

Referring to FIGS. 1 to 3, the wearable device 100 according to the first embodiment may comprise a panel 101 and a control module 140.

The control module 140 may be disposed on one side of the panel 101. The control module 140 may be electrically connected to one side of the panel 101, transmit a signal or command to the panel 101, and receive a signal, such as a biological signal, from the panel 101.

The control module 140 may transmit a biological signal to the panel 101 under the control of a processor (not illustrated) or by itself, and display biological signal information, such as numbers, letters, images, videos, etc., using a plurality of pixels PX1 to PX4 of each of the plurality of blocks 120.

Meanwhile, the control module 140 may comprise a communication unit (not illustrated), and transmit a biological signal to an external device through the communication unit, so that the biological signal may be displayed as biological signal information on the external device. The external device may be any external device having a display function.

The panel 101 may comprise a first substrate 110, a plurality of blocks 120, and a plurality of connection wirings 130.

The first substrate 110 may support the plurality of blocks 120 and the plurality of connection wirings 130. The first substrate 110 may be a stretchable substrate and may be formed of an insulating material that may be bent or stretched. The first substrate 110 may be called an insulating member, an insulating layer, a supporting member, a supporting layer, etc.

For example, the first substrate 110 may be formed of a flexible material. For example, the first substrate 110 may be formed of a silicone rubber such as polydimethylsiloxane (PDMS), an elastic polymer such as polyurethane (PU), polytetrafluoroethylene (PTFE), etc., but is not limited thereto.

A plurality of blocks 120 may be disposed on a first surface of a first substrate 110. A second surface opposite to the first surface of the first substrate 110 may be a surface that contacts the skin of the wrist 500.

Each of the plurality of blocks 120 may comprise a second substrate 127, a light detector 128, and a plurality of pixels PX1 to PX4.

The second substrate 127 may be disposed on the first substrate 110. The second substrate 127 may be less stretchable than the first substrate 110. That is, the second substrate 127 may be relatively more rigid with respect to the first substrate 110. For example, the second substrate 127 may be made of a flexible plastic material, and for example, the second substrate 127 may be made of polyimide (PI), polyacrylate, polyacetate, etc.

The second substrate 127 may be composed of a plurality of layers.

The plurality of blocks 120 may be arranged in a matrix, but is not limited thereto. For convenience, 24 blocks 120 are illustrated in the drawing, but there may be fewer or more than these.

In an embodiment, each of the plurality of blocks 120 may be capable of displaying and measuring biological signal. As a first example, each of the plurality of blocks 120 may display an image. As a second example, each of the plurality of blocks 120 may measure a biological signal. As a third example, each of the plurality of blocks 120 may measure a biological signal while displaying an image.

The light detector 128 and the plurality of pixels PX1 to PX4 may be disposed on the second substrate 127.

For example, the light detector 128 may be disposed in a central region of the second substrate 127, and the plurality of pixels PX1 to PX4 may be disposed in an edge region of the second substrate 127. The plurality of pixels PX1 to PX4 may be disposed around the light detector 128.

Each of the plurality of pixels PX1 to PX4 may comprise a plurality of semiconductor light-emitting elements. For example, each of the plurality of pixels PX1 to PX4 may comprise at least one or more first semiconductor light-emitting element 121, at least one or more second semiconductor light-emitting element 122, and at least one or more third semiconductor light-emitting element 123.

For example, an upper side of each of the first to third semiconductor light-emitting elements 121 to 123, that is, a part of the surface contacting the third substrate 129, may be formed by a layer comprising a reflective pattern, a reflective layer, reflective particles, etc. Accordingly, a portion 151a, 152a, and 153a of the first to third light 151 to 153 emitted from the first to third semiconductor light-emitting elements 121 to 123 may travel forward, and other portion 151b, 152b, and 153b may be reflected by a layer comprising a reflective pattern, a reflective layer, reflective particles, etc. and travel backward. For example, layer comprising the reflective pattern, the reflective layer, the reflective particle, etc., may be disposed only in an edge region or a center region of the upper side of each of the first to third semiconductor light-emitting elements 121 to 123, but is not limited thereto.

The first to third semiconductor light-emitting elements 121 to 123 may be disposed in a row along one direction. The arrangement order of the first to third semiconductor light-emitting elements 121 to 123 may be free. For example, the second semiconductor light-emitting element 122 may be disposed adjacent to the first semiconductor light-emitting element, and the third semiconductor light-emitting element 123 may be disposed adjacent to the second semiconductor light-emitting element 122, but is not limited thereto.

The first semiconductor light-emitting element 121, the second semiconductor light-emitting element 122, and the third semiconductor light-emitting element 123 may be formed of a semiconductor compound material. For example, the first semiconductor light-emitting element 121, the second semiconductor light-emitting element 122, and the third semiconductor light-emitting element 123 may comprise a group II-IV compound or a group III-V compound, but is not limited thereto.

The first semiconductor light-emitting element 121 can emit the first light 151, the second semiconductor light-emitting element 122 can emit the second light 152, and the third semiconductor light-emitting element 123 can emit the third light 153. For example, the first light 151, the second light 152, and the third light 153 can have light of different wavelength bands. For example, the first light 151 can comprise red light, the second light 152 can comprise green light, and the third light 153 can comprise blue light.

An image can be displayed by red light, green light, and blue light emitted from the first to third semiconductor light-emitting elements 121 to 123 of each of the plurality of pixels PX1 to PX4. As in the drawing, when four pixels PX1 to PX4 are provided in each of the plurality of blocks 120, an image may be displayed from each of the four pixels PX1 to PX4, and since this image is implemented on the plurality of blocks 120, a desired image can be freely displayed.

Meanwhile, according to the embodiment, a biological signal can be measured by at least one or more semiconductor light-emitting element among the plurality of semiconductor light-emitting elements of each of the plurality of pixels PX1 to PX4 and the light detector 128. That is, a biological signal measuring unit or a biological signal measuring module can be configured by at least one or more semiconductor light-emitting element among the plurality of semiconductor light-emitting elements of each of the plurality of pixels PX1 to PX4 and the light detector 128. In this instance, the light emitted from the semiconductor light-emitting element constituting the biological signal measuring unit may passe through the blood vessel 501 of the subject, i.e., the wrist 500, and may be received by the light detector 128, thereby allowing the biological signal to be measured. Here, the biological signal may be a blood pressure signal, specifically, a photoplethysmography (PPG) signal.

As a first example, the second semiconductor light-emitting element 122 may be a semiconductor light-emitting element capable of displaying and measuring a biological signal. That is, as illustrated in FIG. 5, a portion 152a of the second light 152 emitted from the second semiconductor light-emitting element 122, that is, the second-first light 152a, may travel forward, and another portion 152b of the second light 152 emitted from the second semiconductor light-emitting element 122, that is, the second-second light 152b, may travel backward. In this instance, the second-first light 152a may travel forward and be used to display an image together with other lights, that is, the first light 151 and the third light 153. The second-second light 152b may travel backward and reflected by the blood vessel 501 of the wrist 500 and detected by the light detector 128.

As a second example, the first semiconductor light-emitting element 121 may be a semiconductor light-emitting element capable of displaying and measuring a biological signal. That is, as illustrated in FIG. 5, a portion 151a of the first light 151 emitted from the first semiconductor light-emitting element 121, that is, the first-first light 151a, may travel forward, and another portion 151b of the first light 151 emitted from the first semiconductor light-emitting element 121, that is, the first-second light 151b, may travel backward. In this instance, the first-first light 151a may travel forward and used to display an image together with other lights, that is, the second light 152 and the third light 153. The first-second light 151b may travel backward and reflected by the blood vessel 501 of the wrist 500 and detected by the light detector 128.

As a third example, the third semiconductor light-emitting element 123 may be a semiconductor light-emitting element capable of display and biological signal measurement. That is, as illustrated in FIG. 5, a portion 153a of the third light 153 emitted from the third semiconductor light-emitting element 123, that is, the third-first light 153a, may travel forward, and another portion 153b of the third light 153 emitted from the third semiconductor light-emitting element 123, that is, the third-second light 153b, may travel backward. In this instance, the third-first light 153a may travel forward and used to display an image together with other lights, that is, the second light 152 and the third light 153. The third-second light 153b may be detected by the light detector 128 by being reflected by the blood vessel 501 of the wrist 500 and traveling backward.

In the above, the light traveling forward may refer to light traveling toward the third substrate 129, and the light traveling backward may refer to light traveling toward the first substrate 110.

As illustrated in FIG. 5, all of the first semiconductor light-emitting element 121, the second semiconductor light-emitting element 122, and the third semiconductor light-emitting element 123 included in the pixels PX1 to PX4 may be used as light sources for measuring biological signal.

In this way, when all of the first semiconductor light-emitting element 121, the second semiconductor light-emitting element 122, and the third semiconductor light-emitting element 123 included in the pixels PX1 to PX4 are used as light sources for measuring biological signal, the biological signal may be measured as an average value of the first-second light 151b of the first semiconductor light-emitting element 121, the second-second light 152b of the second semiconductor light-emitting element 122, and the second-third light 153b of the third semiconductor light-emitting element 123 detected by the light detector 128, and this calculation process may be executed in the control module 140.

Although not illustrated, one or two of the first semiconductor light-emitting element 121, the second semiconductor light-emitting element 122, and the third semiconductor light-emitting element 123 included in the pixels PX1 to PX4 can be used as light sources for measuring biological signal.

Meanwhile, the second substrate 127 of each of the plurality of blocks 120 may have a square shape, and the light detector 128 may have a shape corresponding to the second substrate 127, but is not limited thereto.

For example, the light detector 128 may have a square shape identical to a square shape of the second substrate 127. For example, the light detector 128 may have a circular or other shape different from a square shape of the second substrate 127.

The size of the light detector 128 may be smaller than the size of the second substrate 127. For example, the light detector 128 may be disposed on the central region of the second substrate 127. For example, the light detector 128 may vertically overlap with the central region of the second substrate 127, but may not vertically overlap with the edge region of the second substrate 127.

When the light detector 128 has a square shape, a plurality of pixels PX1 to PX4 may be positioned around corners of the light detector 128. For example, four pixels PX1 to PX4 may be positioned around four corners of the light detector 128 having a square shape.

Meanwhile, a plurality of blocks 120 may each comprise a plurality of fourth semiconductor light-emitting elements 124 on the second substrate 127. The fourth semiconductor light-emitting elements 124 may be used as a light source for measuring a biological signal.

A plurality of fourth semiconductor light-emitting elements 124 may be positioned around the light detector 128. For example, at least one or more fourth semiconductor light-emitting element 124 may be positioned between pixels PX1 to PX4 around the light detector 128 on the block 120.

The fourth semiconductor light-emitting element 124 can emit fourth light 154. The fourth light 154 may only travel backward. The fourth light 154 can comprise near-infrared light. To this end, a layer comprising a reflective pattern, a reflective layer, and reflective particles can be disposed on the upper side of the fourth semiconductor light-emitting element 124

The fourth semiconductor light-emitting element 124 may be transmitted a light detector 128 via the subject, i.e., a blood vessel 501 of a wrist 500, so that a biological signal can be measured. Here, the biological signal can be a glucose signal.

The first semiconductor light-emitting element 121, the second semiconductor light-emitting element 122, the third semiconductor light-emitting element 123, or the fourth semiconductor light-emitting element 124 can have a size of micrometers or less. The first semiconductor light-emitting element 121, the second semiconductor light-emitting element 122, the third semiconductor light-emitting element 123, or the fourth semiconductor light-emitting element 124 may be cylindrical, square, oval, plate-shaped, etc., but is not limited thereto.

As illustrated in FIG. 4, each of the plurality of blocks 120 may comprise a driving unit 132 disposed on a second substrate 127. In the drawing, for convenience, the second substrate 127 may be formed of one layer, but in reality, it may be formed of a plurality of layers. For example, a driving unit 132, which may be formed of at least one or more scan transistor, a driving transistor, at least one or more capacitor, etc., capable of driving each of the pixels PX1 to PX4, the fourth semiconductor light-emitting element 124, and/or the light detector 128, may be disposed between the plurality of layers. The scan transistor, the driving transistor, and the capacitor may be formed using a semiconductor process.

Meanwhile, the control module 140 may be disposed on one side of the first substrate 110.

The control module 140 may comprise a control integrated circuit 141 and a connection terminal 142. The connection terminal 142 of the control module 140 may be electrically connected to a connection terminal (not illustrated) disposed on one side of the first substrate 110.

In the drawing, for convenience, the connection terminal 142 is illustrated as an integral form, but may be formed in a plurality of patterns.

A signal or command output from the control integrated circuit 141 may be transmitted to the first substrate 110 through the connection terminal 142. The signal or command may be transmitted to the driving unit 132 through the connection wiring 130 on the first substrate 110. The driving unit 132 can drive the first to fourth semiconductor light-emitting elements 121 to 124 disposed on each of the plurality of blocks 120 according to the corresponding signal or command.

A light signal detected by the light detector 128 disposed on the block 120, for example, at least one or more light signal among the first to fourth lights 151 to 154, can be transmitted to the control module 140 through the connection wiring 130. The control integrated circuit 141 can obtain a biological signal by calculating the light signal received through the connection terminal 142.

Meanwhile, the connection wiring 130 can electrically connect each of the plurality of blocks 120. Each of the plurality of blocks 120 can be spaced apart from each other by a certain distance. The connection wiring 130 can be disposed on the space. That is, the connection wiring 130 can be disposed on the first substrate 110 between the plurality of blocks 120. One side of the connection wiring 130 can be disposed on one edge of an upper surface of the second substrate 127 via a side surface of the second substrate 127. Although not illustrated, the connection wiring 130 and the driving unit 132 can be electrically connected through a via hole in the second substrate 127.

As described above, considering that the first substrate 110 has a stretchable property and is elongated in the X-axis direction or the Y-axis direction, the connection wiring 130 disposed on the first substrate 110 may have a serpentine shape. For example, when the first substrate 110 is stretched, the connection wiring 130 in a serpentine shape may change from a serpentine shape to a straight shape. For example, when the first substrate 110 is restored to its original shape, the connection wiring 130 in a straight shape may change back to its original serpentine shape.

Meanwhile, the wearable device 100 according to the first embodiment may comprise a third substrate 129. The third substrate 129 may protect a plurality of pixels PX1 to PX4 on a plurality of blocks 120, a fourth semiconductor light-emitting element 124, a light detector 128, etc.

The third substrate 129 may be formed by coating an insulating material on the first substrate 110 and then curing it. The third substrate 129 may be in contact with the first substrate 110, the connection wiring 130, the second substrate 127, the first to fourth semiconductor light-emitting elements 121 to 124, the light detector 128, etc.

The third substrate 129 may be a flexible substrate made of an insulating material that can be bent or stretched. The third substrate 129 may be a flexible substrate, which can reversibly expand and contract. For example, the third substrate 129 may be formed of a molding material such as epoxy or silicone or a rubber material, but is not limited thereto. For example, the third substrate 129 may be formed of the same material as the material of the first substrate 110 or the second substrate 127, but is not limited thereto.

Meanwhile, although not illustrated, a polarizing layer may be disposed on the third substrate 129. The polarizing layer may polarize light incident from the outside of the wearable device 100, thereby reducing external light reflection. In addition, other optical films, etc., other than the polarizing layer, may be disposed on the third substrate 129.

According to an embodiment, a plurality of blocks 120 may be included, each of the plurality of blocks 120 may comprise a light detector 128 and a plurality of pixels PX1 to PX4, each of the plurality of pixels PX1 to PX4 may comprise a plurality of semiconductor light-emitting elements, and at least one or more semiconductor light-emitting element among the plurality of semiconductor light-emitting elements may be configured as a biological signal measuring unit together with the light detector 128, thereby enabling image display and biological signal measurement.

According to an embodiment, the wearable device 100 is configured in a patch type, so that it is portable and may be attached to the subject to measure biological signal.

According to an embodiment, a plurality of blocks 120 capable of biological signal measurement may be disposed over a wide area, so that a plurality of biological signals can be measured from a wide surface of the subject, and based on these plurality of biological signals, biological signals can be measured more accurately, thereby improving reliability.

According to an embodiment, a semiconductor light-emitting element having a size of less than a micrometer may be used as a light source for biological signal measurement, thereby enabling miniaturization.

According to an embodiment, blood pressure signals or glucose signals can be measured by a plurality of semiconductor light-emitting elements and one light detector 128, so that more efficient healthcare can be implemented through measurement of various biological signals.

According to an embodiment, biological signals measured by a plurality of semiconductor light-emitting elements and one light detector 128 can be transmitted to an external device for display, thereby enabling efficient information exchange.

### SECOND EMBODIMENT

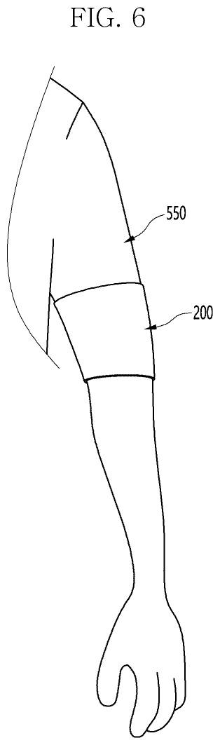

FIG. 6 illustrates a wearable device according to the second embodiment being worn on a forearm.

As shown in FIG. 6, the wearable device 200 according to the second embodiment may be worn on the forearm 550 and can measure various biological signals from the forearm 550. For example, the biological signals may comprise blood pressure, blood glucose, vascular age, arteriosclerosis, vascular elasticity, blood triglycerides, cardiac output, etc.

The wearable device 200 according to the second embodiment may be a cuff-type device.

The cuff-type device may be provided with a member capable of wrapping the forearm 550 at least once, that is, the first and third substrates (110, 129 of FIG. 4) and the expansion/contraction member and sound sensor on the inside thereof, so that a Korotkov sound signal generated after pressure may be applied to the forearm 550 by the expansion/contraction member using the principle of a conventional cuff-type blood pressure monitor may be detected, thereby obtaining a biological signal, such as a blood pressure signal.

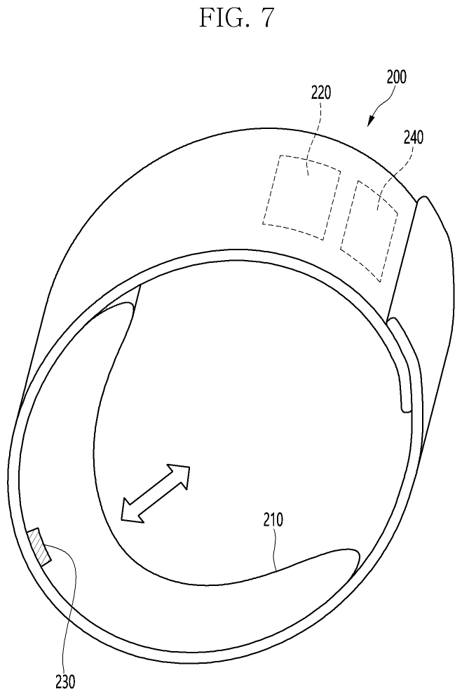

While the first embodiment (FIGS. 1 to 5) detects a biological signal by using a light source and a light detector 128, the second embodiment (FIGS. 6 to 7) detects a biological signal by using the expansion/contraction member 210 and an acoustic sensor 230. Both the first embodiment and the second embodiment may detect blood pressure signals, and for convenience in distinguishing them, the biological signal detected in the first embodiment is named a first blood pressure signal, and the biological signal detected in the second embodiment is named a second blood pressure signal.

Meanwhile, the second embodiment can be combined with the first embodiment. That is, both the first blood pressure signal and the second blood pressure signal can be detected by using the pressure adjustment part 220 and the acoustic sensor 230 of the second embodiment, as well as a plurality of light sources and a light detectors 128 of the first embodiment.

Hereinafter, the second embodiment combined with the first embodiment will be described with reference to FIGS. 2 to 5 and FIG. 7.

FIGS. 2 to 5 and FIG. 7 are perspective views illustrating a wearable device according to the second embodiment.

Referring to FIG. 7, the wearable device 200 according to the second embodiment may comprise an expansion/contraction member 210, a pressure adjustment part 220, and an acoustic sensor 230.

The expansion/contraction member 210 may be mounted on a second surface of the first substrate 110. The expansion/contraction member 210 may be, for example, an air bladder. A second surface may be the opposite side of the first surface that contacts the second substrate 127.

The pressure adjustment part 220 may adjust the pressure of the expansion/contraction member 210 so that pressure is applied to the subject, for example, a forearm 550. For example, the pressure adjustment part 220 may be an actuator or cylinder that injects or exhausts air into the expansion/contraction member 210, but is not limited thereto.

Specifically, the wearable device 200 according to the second embodiment, which is equipped with the first substrate 110 and the third substrate 129, may be wound around the circumference of the forearm 550 and then fastened, as shown in FIG. 6. The wearable device 200 according to the second embodiment may be equipped with a fastening part, and the wearable device 200 according to the second embodiment can be fastened around the circumference of the forearm 550 by the fastening part (not illustrated). The fastening part can be fastened or unfastened.

Thereafter, the expansion/contraction member 210 may be expanded by the driving of the pressure adjustment part 220, so that pressure may be applied to the forearm 550, and the blood vessels of the forearm 550 may be closed. Afterwards, as the pressure applied to the forearm 550 decreases due to the pressure inflated in the expansion/contraction member 210 being driven by the pressure adjustment part 220, the pressure applied to the forearm 550 may also decrease, so that blood flows into the blood vessels of the forearm 550 instantaneously. At this time, the blood flows entangled and turbulent, generating a sound, and then this sound is called Korotkoff's sound.

The Korotkoff sound can be detected as a signal by the acoustic sensor 230. A blood pressure signal, i.e., a second blood pressure signal, can be obtained by the detected Korotkoff sound signal.

The acoustic sensor 230 can be installed at a location where the Korotkoff sound can be detected best. For example, the acoustic sensor 230 can be installed on or within the second surface of the first substrate 110 corresponding to the expansion/contraction member 210, but is not limited thereto. A plurality of acoustic sensors 230 can also be installed.

The second blood pressure signal may be transmitted to the control module 140. The control module 140 may obtain blood pressure information based on the first blood pressure signal obtained in the first embodiment and the second blood pressure signal, and display the blood pressure information through the plurality of pixels PX1 to PX4 of each of the plurality of blocks 120.

As an example, the first blood pressure signal and the second blood pressure signal may be given weights, and the blood pressure information may be obtained based on the first blood pressure signal and the second blood pressure signal to which the weights are respectively reflected.

As another example, the first blood pressure signal and the second blood pressure signal may be given correction values, and the blood pressure information may be obtained based on the first blood pressure signal and the second blood pressure signal to which the correction values are respectively reflected.

According to the second embodiment, by obtaining blood pressure information by considering not only the first blood pressure signal but also the second blood pressure signal, more accurate blood pressure information may be provided, thereby enhancing the reliability of information or products.

For example, in the first embodiment, when the first blood pressure signal is obtained by using at least one of the first to third lights 151 to 153 emitted from the plurality of first to third semiconductor light-emitting elements 121 to 123, an inaccurate first blood pressure signal may be detected due to the light path, light loss, light quantity, etc.

Since the detection of such an inaccurate first blood pressure signal is corrected through a second blood pressure signal comprising a Korotkoff sound signal as in the second embodiment, more accurate blood pressure information can be obtained.

Meanwhile, the wearable device 200 according to the second embodiment may comprise a communication unit 240.

The communication unit 240 may transmit the second blood pressure signal to an external device, so that the signal may be displayed as biological signal information on the external device. The external device may be any external device having a display function.

### THIRD EMBODIMENT

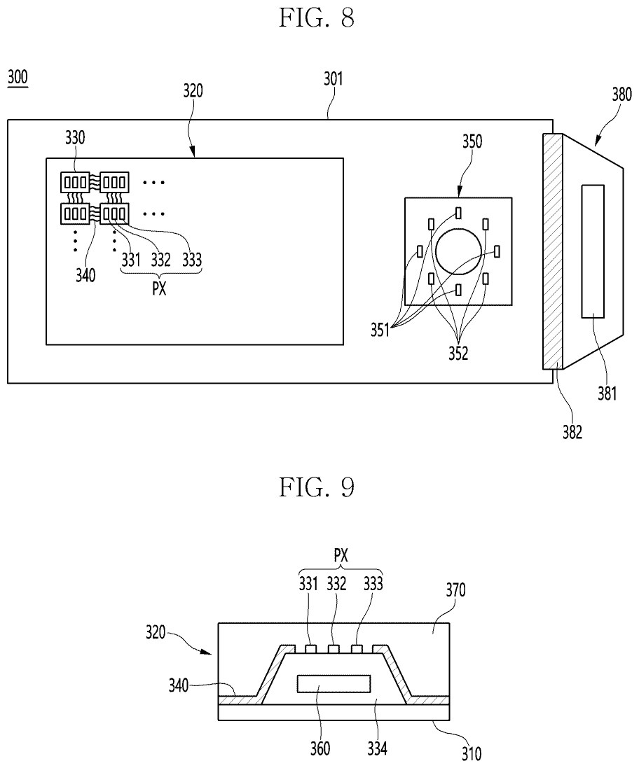

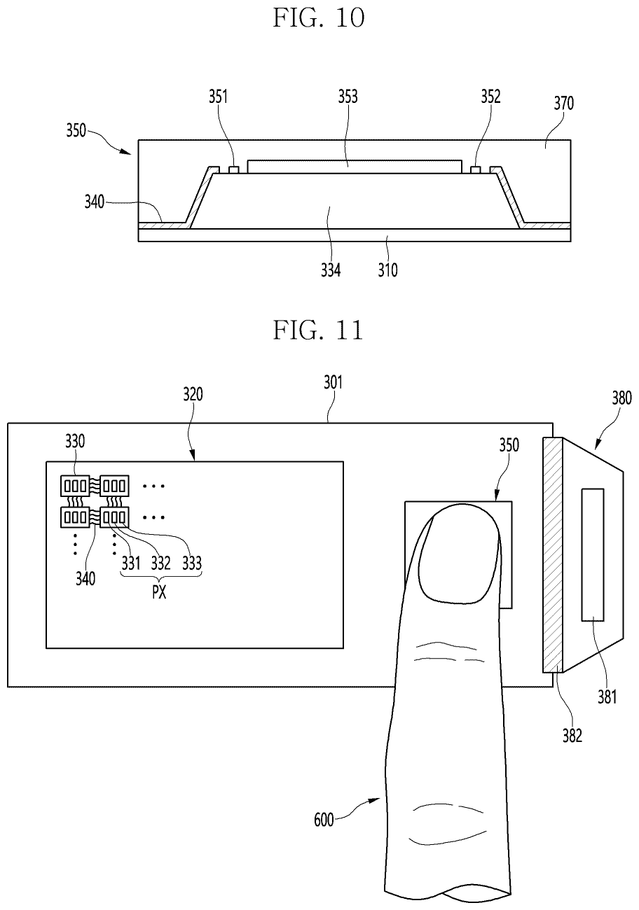

FIG. 8 is a plan view illustrating a wearable device according to a third embodiment. FIG. 9 is a cross-sectional view illustrating pixels in a wearable device according to a third embodiment. FIG. 10 is a cross-sectional view illustrating a biological signal measuring unit in a wearable device according to a third embodiment.

The third embodiment is similar to the first embodiment except that the display unit 320 and the biological signal measuring unit 350 are separated.

That is, in the first embodiment, display and biological signal measurement are possible in each of the plurality of blocks 120, whereas in the third embodiment, the display of the image may be implemented in the display unit 320, and the biological signal measurement may be implemented in the biological signal measuring unit 350.

Referring to FIGS. 8 to 10, the wearable device 300 according to the third embodiment may comprise a panel 301 and a control module 380.

The control module 380 may comprise a control integrated circuit 381 and a connection terminal 382. Since the control integrated circuit 381 and the connection terminal 382 are each described in FIG. 3, a detailed description thereof will be omitted.

The panel 301 may comprise a display unit 320 and a biological signal measuring unit 350.

The display unit 320 may be referred to as a display area, and the biological signal measuring unit 350 may be referred to as a biological signal measuring area. The display unit 320 and the biological signal measuring unit 350 may be disposed on a first substrate 310.

The display unit 320 may comprise a plurality of blocks 330 and a plurality of connection wirings 340. The connection wirings 340 may electrically connect between the plurality of blocks 330.

The plurality of blocks 330 may each comprise a second substrate 334 and a pixel PX. The pixel PX may be disposed on the second substrate 334. In the drawing, one pixel PX is illustrated on the second substrate 334, but two or more pixels may be disposed.

Since each of the plurality of blocks 330 is disposed to be spaced apart from each other, the second substrates 334 of each of the plurality of blocks 330 may also be spaced apart from each other.

The pixel PX may comprise a first semiconductor light-emitting element 331, a second semiconductor light-emitting element 332, and a third semiconductor light-emitting element 333. The first semiconductor light-emitting element 331 may emit a first light 361, the second semiconductor light-emitting element may emit a second light 362, and the third semiconductor light-emitting element 333 may emit a third light 363. For example, the first light 361 may comprise red light, the second light 362 may comprise green light, and the third light 363 may comprise blue light.

Therefore, the display unit 320 may display an image or information through the pixel PX of each of the plurality of blocks 330.

Meanwhile, the biological signal measuring unit 350 may be disposed on one side of the display unit 320. In the drawing, it may be illustrated as being disposed between the display unit 320 and the control module 380, but is not limited thereto.

The biological signal measuring unit 350 may comprise a second substrate 334, a light detector 353, and a plurality of fourth semiconductor light-emitting elements 351.

The second substrate 334 may be disposed on the first substrate 310. The plurality of fourth semiconductor light-emitting elements 351 and the light detector 353 may be disposed on the second substrate 334. The plurality of fourth semiconductor light-emitting elements 351 may be disposed around the light detector 353.

Although the drawing illustrates the second substrate 334 as having a square shape, it may have a circular or other shape. The drawing illustrates that the light detector 353 has a circular shape, but it may have a square shape or other shape.

When the light detector 353 has a circular shape, a plurality of fourth semiconductor light-emitting elements 351 may be disposed along the circumference of the light detector 353.

The fourth semiconductor light-emitting element 351 may emit fourth light 364. The fourth light 364 may be the same as light emitted from one of the first semiconductor light-emitting element 331, the second semiconductor light-emitting element 332, and the third semiconductor light-emitting element 333, but is not limited thereto.

For example, the fourth semiconductor light-emitting element 351 may be a semiconductor light-emitting element that emits green light, similar to the second semiconductor light-emitting element 332 that emits green light.

The fourth light 364 emitted from the fourth semiconductor light-emitting element 351 may travel forward and then be received as a blood pressure signal by the light detector 353 through the subject. To this end, a layer comprising a reflective pattern, a reflective layer, and reflective particles may be disposed on the lower side of the fourth semiconductor light-emitting element 351. The lower side of the fourth semiconductor light-emitting element 351 may have a surface that contacts the second substrate 334.

The incident surface of the light detector 353 may be positioned in the same direction as the emission surface of each of the first to third semiconductor light-emitting elements 331 to 333. That is, the emission surface of each of the first to third semiconductor light-emitting elements 331 to 333 and the incident surface of the light detector 353 may contact the third substrate 370.

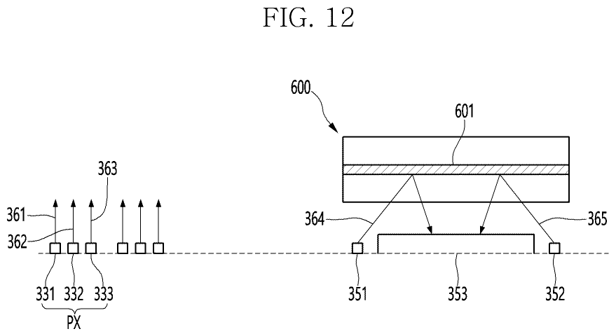

As illustrated in FIGS. 11 and 12, the subject, i.e., a finger 600, may be disposed on a biological signal measuring unit 350. The finger 600 may be pressed downward after coming into contact with the upper side of the biological signal measuring unit 350. In this instance, fourth light 364 emitted from a plurality of fourth semiconductor light-emitting elements 351 and traveled forward may be reflected by a blood vessel 601 of the finger 600 and received by a light detector 353. The light detector 353 may receive a plurality of fourth lights 364 that have passed through the blood vessel 601 of the finger 600 from each of the fourth semiconductor light-emitting elements 351 disposed at different positions. For example, when fourth semiconductor light-emitting elements 351 having four are provided, the light detector 353 may receive fourth lights 364 having four. In this way, the received fourth light 364 having four can be transmitted to the control module 380. The control module 380 can obtain a blood pressure signal using the fourth light 364 having four. For example, the blood pressure signal can be obtained using an average value of the fourth light 364 having four, but is not limited thereto. Here, the blood pressure signal can be a pulse signal.

Meanwhile, the biological signal measuring unit 350 can comprise a plurality of fifth semiconductor light-emitting elements 352.

The plurality of fifth semiconductor light-emitting elements 352 can be disposed around the light detector 353 on the second substrate 334. For example, each of the plurality of fifth semiconductor light-emitting elements 352 can be disposed between the plurality of fourth semiconductor light-emitting elements 351. Accordingly, the fourth semiconductor light-emitting element 351 and the fifth semiconductor light-emitting element 352 may be alternately disposed around the light detector 353.

The fifth semiconductor light-emitting element 352 may emit fifth light 365. For example, the fifth light 365 may comprise near-infrared light.

The fifth light 365 emitted from the fifth semiconductor light-emitting element 352 may travel forward and then be received as a blood pressure signal by the light detector 353 through the subject. To this end, a layer comprising a reflective pattern, a reflective layer, and reflective particles may be disposed on the lower side of the fifth semiconductor light-emitting element 352. The lower side of the fourth semiconductor light-emitting element 351 may have a surface that contacts the second substrate 334.

As illustrated in FIGS. 11 and 12, the subject, i.e., a finger 600, may be disposed on a biological signal measuring unit 350. The finger 600 may be pressed downward after coming into contact with the upper portion of the biological signal measuring unit 350. In this instance, fifth light 365 emitted from a plurality of fifth semiconductor light-emitting elements 352 and traveled forward may be reflected by a blood vessel 601 of the finger 600 and received by a light detector 353. The light detector 353 may receive a plurality of fifth light 365 that have passed through the blood vessel 601 of the finger 600 from each of the plurality of fifth semiconductor light-emitting elements 352 disposed at different positions. For example, when fifth semiconductor light-emitting elements 352 having four are provided, the light detector 353 may receive fifth light 365 having four. In this way, the received fifth lights 365 having four can be transmitted to the control module 380. The control module 380 can obtain a glucose signal using the fifth lights 365 having four. For example, the glucose signal can be obtained using the average value of the four fifth lights 365 having four, but is not limited thereto.

Meanwhile, as illustrated in FIG. 12, all of the first to fifth lights 361 to 365 emitted from each of the first to fifth semiconductor light-emitting elements 352 can travel forward. In this instance, an image can be displayed in front by the first to third lights 361 to 363. When the subject, i.e., a finger 600, is disposed on the biological signal measuring unit 350 in the front, the fourth light 364 and the fifth light 365 may travel forward and be received by the light detector 353 via the finger 600. A blood pressure signal can be detected by the fourth light 364 received by the light detector 353, and a glucose signal can be detected by the fifth light 365.

Meanwhile, the first to fifth semiconductor light-emitting elements 352 can have a size of micrometers or less.

The first substrate 310, the second substrate 334, the third substrate 370, the connection wiring 340, the first semiconductor light-emitting element 331, the second semiconductor light-emitting element 332, the third semiconductor light-emitting element 333, and the driving unit 360 have been described in detail in the first embodiment (FIG. 4), so that a detailed description thereof will be omitted.

### INDUSTRIAL APPLICABILITY

The embodiment can be applied to a portable device capable of measuring a biological signal.

1. A wearable device, comprising:
a first substrate;
a plurality of blocks on a first surface of the first substrate; and
a plurality of connection wirings configured to connect between the plurality of blocks, wherein the plurality of blocks each comprises:
a second substrate on the first substrate;
a light detector on the second substrate; and
a plurality of pixels around the light detector on the second substrate,
wherein the plurality of pixels each comprises:
at least one or more first semiconductor light-emitting element configured to emit a first light;
at least one or more second semiconductor light-emitting element configured to emit a second light; and
at least one or more third semiconductor light-emitting element configured to emit a third light, and
wherein the second semiconductor light-emitting element is configured to emit a portion of the second light forward and another portion of the second light backward to be transmitted to the light detector,
wherein the connection wiring is disposed at an edge region of an upper surface of the second substrate via a side surface of the second substrate from an upper surface of the first substrate,
wherein the light detector is disposed in a central region of the upper surface of the second substrate, and
wherein the first to third semiconductor light-emitting elements of each of the plurality of pixels are disposed on the edge region of the upper surface of the second substrate along the circumference of the light detector.

2. The wearable device of claim 1, wherein the first semiconductor light-emitting element is configured to emit a portion of the first light forward and another portion of the first light backward to be transmitted to the light detector.

3. The wearable device of claim 2, wherein the third semiconductor element is configured to emit a portion of the third light forward, and another portion of the third light backward to be transmitted to the light detector.

4. The wearable device of claim 3, wherein an image is displayed by the first light, the second light, and the third light emitted forward, and
wherein at least one or more of the first light, the second light, or the third light emitted backward is received as a first blood pressure signal by the light detector through a subject.

5. The wearable device of claim 1, wherein the second substrate and the light detector each has a square shape, and
wherein the size of the light detector is smaller than the size of the second substrate.

6. The wearable device of claim 5, wherein the plurality of pixels are positioned around the corners of the light detector.

7. The wearable device of claim 1, wherein the plurality of blocks each comprises:
a plurality of fourth semiconductor light-emitting elements configured to emit fourth light around the light detector on the second substrate.

8. The wearable device of claim 7, wherein the fourth light is received as a glucose signal by the light detector through a subject.

9. The wearable device of claim 7, wherein the plurality of fourth semiconductor light-emitting elements are positioned between the plurality of pixels.

10. The wearable device of claim 7, wherein the first to fourth semiconductor light-emitting elements each has a size of micrometer or less.

11. The wearable device of claim 1, comprising:
a control module on one side of the first substrate.

12. The wearable device of claim 11, comprising:
an expansion/contraction member on a second side of the first substrate opposite the first side;
a pressure adjustment part configured to adjust a pressure of the expansion/contraction member so that pressure is applied to a subject; and
an acoustic sensor configured to detect a second blood pressure signal generated from the subject,
wherein the control module is configured to display blood pressure information obtained based on the first blood pressure signal and the second blood pressure signal on the plurality of pixels.

13. The wearable device of claim 12, comprising:
a communication unit configured to transmit the second blood pressure signal to an external device.

14. The wearable device of claim 1, wherein the first substrate comprises a stretchable substrate.

15. The wearable device of claim 1, wherein the wearable device comprises a patch type or a cuff type.

16. A wearable device, comprises:
a first substrate;
a display unit comprising a plurality of blocks on the first substrate;
a plurality of connection wirings configured to connect between the plurality of blocks; and
a biological signal measuring unit on one side of the display unit on the first substrate,
wherein the plurality of blocks each comprises:
a second substrate on the first substrate; and
a pixel on the second substrate,
wherein the pixel comprises:
at least one first or more semiconductor light-emitting element configured to emit a first light;
at least one or more second semiconductor light-emitting element configured to emit a second light; and
at least one or more third semiconductor light-emitting element configured to emit a third light,
wherein the biological signal measuring unit comprises:
a second substrate on the first substrate;
a light detector on the second substrate; and
a plurality of fourth semiconductor light-emitting elements configured to emit a fourth light around the light detector on the second substrate, and
wherein the fourth light is identical to light emitted from one of the first to third semiconductor light-emitting elements,
wherein the connection wiring is disposed at an edge region of an upper surface of the second substrate via a side surface of the second substrate from an upper surface of the first substrate,
wherein the light detector is disposed in a central region of the upper surface of the second substrate, and
wherein the plurality of fourth semiconductor light-emitting elements are disposed in the edge region of the upper surface of the second substrate along the circumference of the photodetector.

17. The wearable device of claim 16, wherein the fourth light is received as a blood pressure signal by the light detector through a subject.

18. The wearable device of claim 16, wherein the biological signal measuring unit comprises:
a plurality of fifth semiconductor light-emitting elements configured to emit fifth light around the light detector on the second substrate.

19. The wearable device of claim 18, wherein the fifth light is received as a glucose signal by the light detector through a subject.

20. The wearable device of claim 18, wherein the first to fifth semiconductor light-emitting elements each has a size of micrometer or less.
